(19) United States
(12) Patent Application Publication
(10) Pub. No.: US 2007/0277254 A1
(43) Pub. Date: Nov. 29, 2007

(54) Method for Increasing Ploidy in a Plant

(76) Inventor: Graeme David Richards, New South Wales (AU)

Correspondence Address: DYKEMA GOSSETT PLLC, 10 S. WACKER DR., STE. 2300, CHICAGO, IL, 60606, US

(21) Appl. No.: 10/573,340

(22) PCT Filed: Sep. 24, 2004
(86) PCT No.: PCT/AU04/01310

§ 371 Date: Feb. 21, 2007

(30) Foreign Application Priority Data
Sep. 26, 2003 (AU) 2003905278

(51) Int. Cl. A01H 5/00; A01N 37/18

(52) U.S. Cl. 800/260; 800/276; 504/339

(57) ABSTRACT

Abstract: The present invention provides a method of increasing ploidy in cells of a woody perennial plant, the method comprising: contacting plant tissue comprising dividing cells with an effective amount of a composition comprising about 0.5% w/v colchicine to about 3% w/v colchicine. Woody perennial plants with increased ploidy obtained by the method of the invention are also provided.

### TECHNICAL FIELD

The present invention relates generally to the field of plant breeding and the production of plants and plant breeding lines comprising improved or new desirable traits. In particular, the invention relates to methods of increasing the ploidy of a plant species or plant variety. The invention also relates to plant varieties and species produced thereby and to methods of generating new plant varieties comprising at least one desirable trait.

### BACKGROUND OF THE INVENTION

The manipulation of ploidy in plant tissue has been used, for example, to produce fruit without seeds, to introduce fertility into hybrids that otherwise would not normally be able to be created, and to produce plants with other desirable characteristics, such as, in flowering plants, larger blooms or more intense colour.

Plant breeders use a naturally occurring compound, colchicine, which inhibits the development of spindle fibres during nuclear or cell division, to increase the chromosome number (for example, doubling up the DNA of a species). Then, with a matched chromosome number, new hybrids can be produced that could not otherwise be created.

Current methods used for increasing chromosome number in plants generally employ colchicine concentrations in the order of 0.001% w/v to 0.2% w/v. The percentage of cells of the exposed plant tissue in which chromosome multiplication has been effected by known methods is generally very low. Furthermore, the reliability of the known methods, in terms of reproducibly attaining even low percentages of cells in which ploidy has increased after exposure is variable. As a result, production of, for example, new breeding lines or plants having desirable traits by the known methods is inefficient.

### SUMMARY OF THE INVENTION

It has surprisingly been found that exposure of plant tissue to relatively high concentrations of colchicine results in improved yield of cells in which chromosome multiplication has been effected. It has also surprisingly been found that improved yields may be attained more reproducibly than is attained with known methods. In addition, the yield of cells having an increase in ploidy may be improved when exposure of the plant tissue to the agent capable of inhibiting spindle formation is commenced substantially coincidental with the breaking of dormancy of the plant tissue. That is, when the plant tissue or plant is put in an active state with respect to cell division.

Accordingly, in a first aspect, the present invention relates to a method of increasing ploidy in cells of a woody perennial plant, the method comprising:

contacting plant tissue comprising dividing cells with an effective amount of a composition comprising an agent capable of inhibiting spindle formation, wherein said contacting commences substantially coincidental with breaking dormancy of said plant tissue.

According to an embodiment of the methods of the invention, the plant tissue may be at least one bud grafted onto a rootstock plant. The apical shoot and all buds of the rootstock plant may be removed, so as to allocate more of the plant resources to the grafted bud(s). The plant tissue may be a single grafted bud.

According to another embodiment of the methods of the invention, the plant tissue may be exposed to ultraviolet, or fluorescent light or to a mercury and/or sodium lamp substantially continuously subsequent to said contacting at least until growth from the treated tissue occurs.

Accordingly, in a further aspect, the present invention relates to a method of increasing ploidy in cells of a woody perennial plant, the method comprising:

contacting plant tissue comprising dividing cells with an effective amount of a composition comprising about 0.5% w/v colchicine to about 3% w/v colchicine.

In a further aspect, the present invention relates to a method of increasing ploidy in cells of a deciduous woody perennial plant, the method comprising:

contacting at least one bud of said plant, wherein said bud comprises actively dividing cells, with a composition comprising about 0.5% w/v colchicine to about 3% w/v colchicine,

at least partially enveloping said bud with a material capable of inhibiting gaseous exchange, wherein said contacting is substantially continuous over a period of from about 5 days to about 15 days.

In a further aspect, the present invention relates to a method of generating a plant having a desired ploidy level, the method comprising:

contacting plant tissue comprising dividing cells with an effective amount of a composition comprising about 0.5% w/v colchicine to about 3% w/v colchicine,

generating at least one plant from tissue so contacted, and

selecting at least one plant having the desired ploidy level.

In a further aspect, the present invention relates to a method of generating a plant, the method comprising:

contacting plant tissue comprising dividing cells with an effective amount of a composition comprising about 0.5% w/v colchicine to about 3% w/v coichicine, selecting plant tissue of increased ploidy level,

generating at least one plant from said selected plant tissue, and

crossing said generated plant with a plant of the same or different ploidy level.

In a further aspect, the present invention relates to a method of generating a plant having at least one desired trait, the method comprising:

contacting plant tissue comprising dividing cells with an effective amount of a composition comprising about 0.5% w/v colchicine to about 3% w/v colchicine, selecting plant tissue of increased ploidy level,

generating at least one plant from said selected plant tissue,

crossing said generated plant with a plant of the same or different ploidy level, and

selecting at least one progeny plant having the desired trait.

In a further aspect, the present invention relates to a method of generating a plant having at least one desired trait, the method comprising:

contacting parental diploid plant tissue comprising dividing cells with an effective amount of a composition comprising about 0.5% w/v colchicine to about 3% w/v colchicine,

selecting tetraploid tissue from said treated plant tissue,

generating at least one tetraploid plant from said tetraploid tissue,

crossing said tetraploid plant with a diploid plant, and

selecting at least one progeny plant having the desired trait.

In a further aspect, the invention relates to a plant or propagative material thereof, of fruit thereof, produced by a method of the invention.

### DETAILED DESCRIPTION AND EXAMPLES

It has surprisingly been found that the use of hitherto believed excessive concentrations of an agent capable of inhibiting spindle formation is advantageous in inducing increased ploidy in plant tissue. The agent capable of inhibiting spindle formation in the plant tissue may be any suitable agent, for example colchicine, oryzalin (Surflan™), trifluralin, amiprophos-methyl, and N₂O gas. It is also envisaged that a combination of agents may be used.

Where the agent capable of inhibiting spindle formation is colchicine, the colchicine may be administered as a composition comprising about 0.5% w/v colchicine to about 3% w/v colchicine. Thus, the composition may comprise colchicine in a concentration of about 0.5% w/v, about 0.6% w/v, about 0.7% w/v, about 0.8% w/v, about 0.9% w/v, about 1% w/v, about 1.1% w/v, about 1.2% w/v, about 1.3% w/v, about 1.4% w/v, about 1.5% w/v, about 1.6% w/v, about 1.7% w/v, about 1.8% w/v, about 1.9% w/v, about 2% w/v, about 2.1% w/v, about 2.2% w/v, about 2.3% w/v, about 2.4% w/v, about 2.5% w/v, about 2.6% w/v, about 2.7% w/v, about 2.8% w/v, about 2.9% w/v or about 3% w/v.

Where the agent capable of inhibiting spindle formation is oryza the oryzalin may be administered as a composition comprising about 0.001% w/v oryzalin, about 0.005% w/v oryzalin, about 0.01% oryzalin, about 0.05% w/v oryzalin, 0.1% w/v oryzalin, or about 0.5% w/v oryzalin.

Contact of the plant tissue with the agent, for example colchicine, may be commenced substantially coincidental with, or soon after the plant tissue has broken dormancy. This may be referred to as priming the plant or plant tissue before, or substantially coincidental with, contact with the agent, such that the growing point is contacted substantially at the earliest time of activity. For example, where the plant tissue is one or more buds on a rootstock, the rootstock may be exposed to conditions sufficient to break dormancy prior to contact with the composition. Conditions sufficient to break dormancy will depend on the particular plant and may be determined by methods known to those of skill in the art. For example, a plant having a particular chill requirement may be maintained at an appropriate temperature for a time sufficient to satisfy the chill requirement and then exposing the plant to an appropriate (warmer) temperature for a time sufficient to prime bud break. For example, Prunus salicina low chill 2N=16, chill at 5° C. for 200 hours; Prunus domestica (2N=48), chill at 5° C. for 1200 hours, then hold at 22° C. to break dormancy.

Similarly, it is envisaged that the method is also applicable to increasing the ploidy of cells in grafted plant tissue. The grafted tissue or scion may have different requirements for breaking dormancy compared with the rootstock, for example the rootstock may have a lower chill requirement than does the scion or the rootstock may have a higher chill requirement than the scion.

One or more grafted buds may be used. Removal of all buds and/or growing shoots from the rootstock, before or once the bud(s) have taken, will ensure that an increased proportion of the plant resources will be directed towards the grafted bud(s), increasing the likelihood of cells surviving the treatment and mutating. Use of a single grafted bud on the rootstock, which will therefore be ‘apically dominant’ will further increase the chances of survival and mutation of grafted cells. In order to reduce the amount of time required to break dormancy of the plant, bud breaking agents such as hydrogen cyanimide may be employed or treatment such as exposure to ultraviolet or fluorescent light or mercury and/or sodium vapour lamp(s) may be used.

Exposure of treated plant tissue to ultraviolet or fluorescent light or mercury and/or sodium vapour lamp(s) substantially continuously during and/or subsequent to treatment with colchicine (that is, not allowing a nocturnal phase) may also be carried out so as to encourage growth, cell division and therefore increase the likelihood of successful mutation of the plant cells. Ideally, although not necessarily, exposure is carried out at least until growth from the treated tissue occurs.

Contacting the plant tissue with colchicine, or any other suitable agent capable of inhibiting spindle formation, may be effected by any suitable means, such as by substantially immersing or substantially submersing the plant tissue into the composition, for example by dipping the plant tissue into the composition, or by dripping or dropping the composition onto the plant tissue, for example by use of a pipette, dropper or syringe, or by spraying the composition onto the plant tissue, or by painting the plant tissue with the composition, such as by an appropriately sized paintbrush, cloth or cotton bud. The composition may also be administered to the plant tissue by injection, for example by use of a hypodermic-type syringe.

The composition may be administered to the plant tissue before or after the plant tissue has been at least partially enveloped in an absorbant material. The absorbant material may be any suitable absorbant material, for example, the absorbant material may be laboratory standard cotton or cotton wool, sponge, foam. For example, the plant tissue may be at least partially enveloped in an absorbant material and then the composition administered by any of the above-described means such that the absorbant material becomes at least partially saturated with the composition. The composition may be administered such that the absorbant material becomes saturated with the composition. Administration of the composition to the absorbant material may be described as indirect administration of the composition to the plant tissue. Administration of the composition to the plant tissue may be indirect or direct administration.

The composition may be in any suitable form. For example, the composition may be in the form of a solution, paste, or salve. The composition may be in the form of an aqueous solution.

After administration of the composition to the plant tissue, the plant tissue, which may or may not be at least partially enveloped in an absorbant material, may be at least partially enveloped with a material capable of inhibiting gaseous exchange. The material capable of inhibiting gaseous exchange may be capable of partially, substantially completely or completely inhibiting gaseous exchange. For example, the material capable of inhibiting gaseous exchange may be a plastic film, for example in the form of a bag. Thus, for example, the plant tissue may be at least partially enveloped in an absorbant material, to which the composition comprising the agent capable of inhibiting spindle formation, such as colchicine, is administered in an amount sufficient to at least partially or completely saturate the absorbant material, before the absorbant material is at least partially enveloped in a plastic film or bag.

The agent capable of inhibiting spindle formation may be administered in combination with at least one additional agent capable of enhancing penetration of the spindle formation inhibiting agent into the plant tissue. These additional agents may collectively or individually be referred to, for the purposes of the present invention, as a carrier(s). Suitable carriers include, for example, surfactants, wetting agents, oils and dimethylsulfoxide. The oil may be, for example a non-phytotoxic oil or a phytotoxic oil used in a non-toxic amount. Where a carrier is used a lower concentration of the agent capable of inhibiting spindle formation, such as colchicine, may be used compared to the absence of a carrier. A combination of different types of carrier(s) may also be used.

The carrier(s) may be administered simultaneously with the agent capable of inhibiting spindle formation, such as by contacting the plant tissue with a composition comprising an agent capable of inhibiting spindle formation and one or more carriers, or by sequential administration of the carrier(s) and the agent capable of inhibiting spindle formation. When administered sequentially, the carrier(s) and the agent capable of inhibiting spindle formation may be administered to the plant tissue in any order, for example administration of the carrier(s) to the plant tissue prior to administration of the agent capable of inhibiting spindle formation or by administration of the agent capable of inhibiting spindle formation to the plant tissue prior to administration of the carrier(s). When administered sequentially, the carrier(s) and the agent capable of inhibiting spindle formation are administered over a time period which provides for overlapping effect.

Substantially continuous contact of the plant tissue with the agent may be achieved by a single administration of the composition or by multiple administrations of the composition to the plant tissue. In this manner the concentration of the agent capable of inhibiting spindle formation may be maintained at or near an optimum level. For example, fresh applications of the composition may be administered one, two, three, four or more times per day for the period of contact of the plant tissue with the agent. Where multiple administrations of the composition are undertaken, the absorbant material at least partially enveloping the plant tissue may or may not be removed and may or may not be replaced as part of the multiple administration(s).

The plant tissue subject to the method of the invention may be maintained under conditions which optimise cell division or growth. Where the plant tissue subject to the method of the invention is maintained under conditions of a naturally-occurring diurnal cycle, which conditions may be natural or artificially induced or simulated, at least one of the administrations of the composition may be administered at a time in the diurnal cycle when cell division is relatively high. For example, at least one administration of the composition may occur early in the morning. Where multiple administrations occur over two or more days, at least one of each administration on each day may occur early in the morning.

Where the plant tissue is maintained under continuous lighting to encourage continuously elevated cell division, administration of the composition may be evenly spaced, for example once every 2, 4, 6, 8, 10, 12, 14, 16, 18, 20, 22 or 24 hours.

In the method of the invention the plant tissue to which the agent capable of inhibiting spindle formation is applied, which may be the original growing point of the plant, may be killed during contact with the agent. Apical side buds may be produced adjacent to the killed main bud. The apical side buds may be mutated, such as by increased ploidy compared to the original plant tissue.

Plant tissue may be assessed for increase in ploidy by any suitable method known to those skilled in the art. For example, increase in ploidy may result in increased cell size that brings about thicker, broader leaves and larger flowers and fruit, shoots of plants having increased ploidy may be thicker and may have shortened internodes and wider crotch angles. As further examples, polyploidy may be evidenced by larger pollen size, or by greater number of chloroplasts per guard cell, or by larger guard cells and stomates. Depending on the type of tissue being analysed for increase in ploidy, methods including root tip squashes, pollen mother cell squashes, pollen grain size and germinal pore counts, stomata size and density determination, and gross morphology may also be used. Appropriate methods for chromosome staining and counting are also known in the art. Advanced techniques such as measurement of the nuclear DNA content of the plant cells, such as by flow cytometry, or microspectrophotometry may be used for ploidy determination.

The method of the invention is suitable for increasing the ploidy of numerous plant species. For example, the method may be applied to any woody perennial plant. The woody perennial plant may be deciduous or evergreen. Examples of deciduous woody perennial plants to which the method may be applied are plants of the genus Prunus. Plants of the genus Prunus to which the method of the invention may be applied include, for example, P. mira, P. mandschurica, P. ansu, P. davidiana, P. brigantiaca, P. ceracifera, P. mume, P. domestica, P. salicina, P. arneniaca, P. simonii, P. americana, P. sibirica, P. mexicana, P. hortulana, P. angustifolia, P. munsoniana, P. umbellata, P. communis, P. persica, P. persica var nectarina, P. pumila, P. besseyi, P. humilis, P. ceracoides, P. avium, P. pseudoceracus, P. campanulata. Where such plants may be usefull in a breeding program but have varying chromosome numbers, the method of the invention may be used to increase, for example to double, the chromosome number. For example, Prunus salicina (2N=16); Prunus avium (2N=16); Prunus pseudoceracus (2N=32). For example, the method of the invention may be used to generate tetraploid tissue or a tetraploid plant from Prunus avium which may then be crossed with Prunus pseudoceracus. For example, the method may be used in assisting a breeding program of crossing a European sweet cherry with 16 chromosome pairs with another species with 32 chromosome pairs. Examples of other genera to which the method of the invention may be applied include Pyrus (pear), such as P. pyrifolia and P. communes and Malus (apple), such as M. domestica, M. asiatica, and M. formosana and citrus, such as C. medica, C. limonia, C. sinensis, C. grandis, C. paradisii C. ichangensis, C. aurantifolia, C. mitis, C. nobilis, Poncirus trifoliata, Persea (avocado), Mangifera (mango), Punica (pomegrante), Olea (olive).

The ability to improve the generation of plant tissue having increased ploidy levels, that is, the ability to improve the generation of polyploid plant tissue and therefore plants, may permit the skilled person to more reliably address issues associated with plant breeding and the generation of new plant species and varieties having desirable characteristics. For example, differences in ploidy levels or chromosome number in prospective parental plants constitutes a difficulty in generating progeny, which may be substantially overcome by manipulating the ploidy levels of the prospective parental plant(s) prior to hybridisation. Manipulation of the ploidy level or chromosome number may or may not equalise the ploidy level or the chromosome number of the prospective parents in order to substantially overcome difficulty in generating progeny. For example, a pollen mixture or polymix may be used to improve the likelihood of successful hybridisation where differences in the ploidy level or chromosome number remain after the method of the invention. Alternatively, the method may be used to restore fertility in a plant variety or cultivar having desired traits. For example, the plant variety having desired traits may be the product of hybridisation between plants of a different species or genera and, due to the failure of the chromosomes to pair correctly in meiosis, will often be sterile. Restoration of fertility in such a variety may be accomplished by doubling the chromosome number. As a further example, it may be desirable to create sterile cultivars of a species, such as in the situation where it is desirable to limit the ability of an important agricultural, commercial or nursery species or variety to reproduce and spread. For example, doubling the chromosome number of a plant may result in sterility due to multiple homologous chromosomes and resultant complications in meiosis. Alternatively, or in addition, sterile triploid plants may be created by hybridisation of a tetraploid with a diploid. In commercial applications where the plant variety or species is a fruiting plant, this highlights a further use to which the method of the invention may be applied, that being, the generation of seedless (or substantially seedless) fruit. The method may also find use in the development of plant varieties having enhanced pest resistance and stress tolerance. For example, increasing the chromosome number and related gene dose has been known to enhance the expression and concentration of secondary metabolites and defence chemicals of the plant.

The use of pollen mixtures obtained from a number of woody perennial varieties, species, or genera may be used to increase the likelihood of successful hybridisation, particularly where the seed parent and the pollen parent are of different species or incompatibility groups, such as occur in cherries (Prunus avium, and related species). Use of pollen mixtures may also be used in overcoming self-incompatibility such as occurs in, for example, plums (for example Prunus salicina and P. domestica), apricots (P. armeniaca) and almonds (P. amygdalus).

The skilled addressee will be aware that there are a number of possibilities for use of the treated plant tissue. For example, the directly treated tissue or apical side buds may be permitted to develop to a stage where they can be assessed for increase in ploidy. The buds may be permitted to continue to develop in situ or one or more buds may be excised and engrafted to one or more alternative rootstock(s). The buds, either in situ as treated or engrafted, may be permitted to develop to maturity, for example to flowering or fruiting stage. The method of the invention thus provides a method for the generation of new plant varieties, cultivars and breeding lines.

As described above, the method of the invention may also be used in the production of a substantially seedless plant variety. For example, this is advantageous in the production of commercially important fruit crops, such as stone fruit or citrus, avocado, mango, or olive. For example, plant tissue of a diploid parental plant having one or more desirable characteristics, such as flesh colour, sugar levels, skin colour, acidity, disease resistance, fruit size, maturity time may be subjected to the method of the invention and resultant tetraploid plant tissue selected. The tetraploid plant tissue is allowed to develop to maturity, either in situ or after excision and engrafting, and may then be hybridised or backcrossed with the original diploid parent plant. The triploid progeny will be substantially seedless. The original parental plant may be either polyembryonic or monoembryonic.

The present invention will now be described, by way of example only, with reference to the following examples, which are not to be taken to be limiting to the scope or spirit of the invention in any way.

### Example 1

Firstly, the plants are budded (on a selected root stock), then they are held in a dormant state and the appropriate chill requirement is given in a cool room (if required). The plants are then removed to a warm environment to prime the bud (which is still dormant). This is continued up to the point at which dormancy is “broken”, cell division is commenced. The bud is then wrapped in absorbent substance, the absorbent material is injected with a 1% aqueous solution of the colchicine drug. Note that this is 10× higher concentration than normal treatments. A plastic surround is used to enclose the bud, to keep it moist but exposed to the drug. This treatment is repeated twice daily for five days minimum. The original growing point is generally killed in the process, however, apical side buds are produced adjacent to this killed main bud, which are usually mutated.

In a next step, to determine whether a ploidy increase has resulted, the stomata on leaves are counted per unit area and compared to the parent. Unexpectedly, it is observed that between 10-20% of the apical buds have their ploidy level increased as a result of this process (compared with only approx 1% in normal techniques). Thus, for example, in citrus, plant leaves become shorter, thicker, wider and oil glands become more prominent.

A chromosome count can be made on the tissue to establish the degree of ploidy. The doubled (or trebled, etc.) numbers of chromosome pairs in desired plant species can then be used for effective hybridising. Interspecific-crosses or intergeneric-crosses are then made more efficiently possible as a result of this method invention.

### Example 2

Polyploidy—Growing Seedless Citrus

In general, all types of citrus (for example, grapefruit, lemon, orange, mandarin, etc) are poly-embryonic. Poly-embryonic means that a seed will germinate with multiple shoots, as opposed to normal seeds from other species, which send-up one sexual shoot (mono-embryonic). Only one of these shoots is a sexual shoot.

Crossing two citrus plants (say “A” and “B”) normally results in a poly-embryonic seed with the same phenotype as one of its parents (say “A”). This may be referred to as clonal to the female parent.

The poly-embryonic shoots germinate from the nucellar tissue (sometimes called placental tissue) of the seed that surrounds the fertilised embryo. This phenomenon is referred to as apomixis. This is a form of natural cloning and is undesirable in breeding as the nucellar tissue interferes with the developing hybrid embryo. The consequence is that viable hybrid crosses are rarely produced. In approximately 2 out of 10,000 seeds, the embryo does germinate and produce a hybrid. The hybrid is inferior to the clonal seedlings in vigour.

To increase the chance of direct hybrids being created, the available germplasm needs to be screened for mono embryonic (˜ is 10% of all germplasm). The mono embryonic is chosen with as many desirable traits to be the female parent, and is crossed with poly or mono as the male. This will result in hybrid progeny.

To produce what is marketed as “seedless” the current state of the art is to grow a mono in isolation from other varieties. This is undesirable due to pressure on growing areas.

Current methods known to breeders do not use poly as a female parent.

The production of triploids as described herein provides improved methods of developing desirable new plants. A mono with desirable attributes is identified. Using the method of the present invention the mono is converted from diploid to tetraploid (i.e. 4N). The tetraploid is crossed back to the initial diploid i.e. the original parent—a form of in breeding. The resulting mono triploid embryo is infertile and seedless.

[image]

Take a single bud and bud this onto a vigorous rootstock in a pot (20 cm). Bud the bud (chip budding) about 10cm above ground and/or where root stock has about 7.5 mm (¾ cm) diameter. Place in 20-25° C. green house for 4-6 weeks and maintain plant with best horticultural practices (i.e. water, fertilise). Once “taken”, cut the rootstock plant about 5-10 mm (½-1 cm) above the bud and remove excess rootstock, giving grafted bud terminal dominance. Increase temperature in greenhouse to 28-38° C. with other optimum growth condition until the bud has started to swell (“move”).

Encapsulate the bud in laboratory standard absorbent material by wrapping over bud and stem and fastening by plastic spring loaded clip around the excess behind the stem. At about 10 am using a pipette, inject cotton wool with 1% aqueous solution of colchicine until cotton wool is saturated around the bud. Wrap in plastic bag to stop evaporation of colchicine.

Repeat the administration procedure at about 3 pm. Repeat the twice daily administration procedure for 5 days. Remove bag and cotton wool and allow the treated plant to grow. Assess morphological features to identify polyploid tissue. If tetraploid then cross with a diploid if, for example, seedlessness is desired. If not, it may be crossed with another plant of the same ploidy level to generate a cross that would not normally, in the absence of the method to increase ploidy level, be achieved.

When plant tissue has been confirmed as tetraploid (i.e. 4N) use on one of the plants:
if mono→female
if poly→male

Back cross to the same variety diploid, or any other diploid. Progeny are then planted out and eventually assessed for desirable trait(s).

It will be appreciated that, although specific embodiments of the invention have been described herein for the purpose of illustration only, various modifications may be made without deviating from the spirit and scope of the invention as defined in the following claims.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. A method of increasing ploidy in cells of a woody perennial plant, the method comprising:
contacting a bud of said plant substantially continuously over a period of at least 1 day with an effective amount of a composition comprising from about 0.5% w/v colchicine to about 3% w/v colchicine, wherein said bud comprises actively dividing cells and is apically or terminally dominant; and
exposing the treated bud to substantially constant illumination until growth from the treated tissue occurs.

41. The method of claim 40, wherein said bud is a bud that has been grafted onto a rootstock plant.

42. A method of increasing ploidy in cells of a woody perennial plant, the method comprising:
contacting a bud of said plant substantially continuously over a period of at least 1 day with an effective amount of a composition comprising from about 0.5% w/v colchicine to about 3% w/v colchicine, wherein said bud has been grafted onto a rootstock plant, comprises actively dividing cells and is apically or terminally dominant.

43. The method of claim 42, further comprising the step of exposing the treated bud to substantially constant illumination until growth from the treated tissue occurs.

44. The method of claims 41 or 42 wherein the apical shoot and all buds of the rootstock plant have been removed.

45. The method of claims 40 or 42, wherein said bud is the sole bud on said plant.

46. The method of claims 40 or 42 wherein the concentration of colchicine is about 1% w/v.

47. The method of claims 40 or 42 wherein the plant is selected from the group consisting of a Prunus spp. Pyrus spp, Malus spp, Citrus spp, Poncirus spp, Persea spp, Mangifera spp, Punica spp, and Olea spp.

48. The method of claims 40 or 42 wherein the method further comprises prior to said contacting step exposing said plant tissue to conditions sufficient to break dormancy of said bud.

49. The method of claim 48 wherein conditions sufficient to break dormancy of said bud comprise maintaining said bud at an appropriate temperature for a time sufficient to satisfy the chill requirement of said bud, optionally in the presence of hydrogen cyanimide, and maintaining said bud at an appropriate temperature for a time sufficient to prime cell division in said bud.

50. The method of claims 40 or 42 wherein said contacting comprises at least partially enveloping said bud with an absorbent material.

51. The method of claims 40 or 42 wherein said bud, and surrounding tissue, is at least partially enveloped with a material capable of inhibiting gaseous exchange.

52. The method of claims 40 or 42 wherein said contacting comprises substantially continuous exposure of said tissue to said composition over a period from about one day to about 30 days.

53. The method of claims 40 or 42 wherein said contacting comprises substantially continuous exposure of said tissue to said composition over a period of about 10 days.

54. The method of claims 40 or 42 wherein said contacting comprises multiple applications of said composition.

55. The method of claim 54 wherein said multiple applications comprise two or more applications per day.

56. The method of claim 54 wherein at least one of said applications is administered when plant cell division is substantially maximal.

57. The method of claims 40 or 42 wherein said bud is exposed to ultraviolet, or fluorescent light or a mercury and/or sodium lamp.

58. The method of claim 57, wherein said plant tissue is exposed to ultraviolet, or fluorescent light or to a mercury and/or sodium lamp prior to said contacting as well as substantially continuously subsequent to said contacting at least until growth from the treated tissue occurs.

59. The method of claims 40 or 42, wherein plant tissue arising from the treated bud, or cells at the base of the bud are checked for ploidy, tissue having the desired ploidy level selected, and the plants grown from the selected tissue is/are tested for inheritability of the desired ploidy level.

60. A method of increasing ploidy in cells of a woody perennial plant, the method comprising:
contacting at least one apically or terminally dominant bud of said plant, wherein said bud comprises actively dividing cells, with a composition comprising about 0.5% w/v colchicine to about 3% w/v colchicine,
at least partially enveloping said bud with a material capable of inhibiting gaseous exchange, wherein said contacting is substantially continuous over a period of from about 5 days to about 15 days; and
exposing the treated bud to substantially constant illumination until growth from the treated tissue occurs.

61. The method of claim 60, wherein said bud has been grafted onto a rootstock plant.

62. A method of increasing ploidy in cells of a woody perennial plant, the method comprising:
contacting at least one bud of said plant with a composition comprising about 0.5% w/v colchicine to about 3% w/v colchicine, wherein said bud has been grafted onto a rootstock plant, is apically or terminally dominant and comprises actively dividing cells; and
at least partially enveloping said bud with a material capable of inhibiting gaseous exchange, wherein said contacting is substantially continuous over a period of from about 5 days to about 15 days.

63. The method of claim 62, further comprising the step of exposing the treated bud to substantially constant illumination until growth from the treated tissue occurs.

64. The method of claims 60 or 62, wherein said bud is the sole bud on said rootstock plant.

65. The method of claims 60 or 62 wherein the concentration of colchicine is about 1% w/v.

66. The method of claims 60 or 62 wherein the plant is selected from the group consisting of Prunus spp. Pyrus spp, and Malus spp.

67. The method of claims 60 or 62 wherein the method further comprises prior to said contacting step exposing said plant tissue to conditions sufficient to break dormancy of said bud.

68. The method of claims 60 or 62, further comprising the steps of:
generating at least one plant from a bud so contacted; and
selecting at least one plant having a desired ploidy level.

69. The method according to claim 68 wherein the desired ploidy level is diploid (2N), tetraploid (4N) or hexaploid (6N), octoploid (8N), decaploid (10N) or dodecaploid (12N).

70. A method of generating a woody perennial plant having at least one desired trait, the method comprising:
generating tetraploid plant tissue from a bud of a diploid parental woody perennial plant variety by a method of any one of claims 40, 42, 60 or 62;
generating at least one tetraploid plant from said tetraploid tissue;
crossing said tetraploid plant with a diploid plant;
generating at least one progeny plant having the desired trait.

71. The method of claim 70 wherein the desired trait is seedlessness.

72. The method of claim 70 wherein said tetraploid plant is back-crossed with said diploid parental woody perennial plant variety.

73. The method of claims 40, 42, 60, or 62, wherein said contacting commences substantially coincidental with breaking dormancy of said bud.

74. A method of increasing ploidy in cells of a woody perennial plant, the method comprising:
contacting a bud of said plant substantially continuously over a period of at least 1 day with an effective amount of a composition comprising an agent capable of inhibiting spindle formation, wherein said contacting commences substantially coincidental with breaking dormancy of said plant tissue, wherein said bud comprises actively dividing cells and is apically or terminally dominant; and
exposing the treated bud to substantially constant illumination until growth from the treated tissue occurs.

75. A method of increasing ploidy in cells of a woody perennial plant, the method comprising:
contacting a bud of said plant substantially continuously over a period of at least 1 day with an effective amount of a composition comprising an agent capable of inhibiting spindle formation, wherein said contacting commences substantially coincidental with breaking dormancy of said plant tissue, wherein said bud is grafted onto a rootstock plant, comprises actively dividing cells and is apically or terminally dominant.

76. The method of claim 75, further comprising the step of exposing the treated bud to substantially constant illumination until growth from the treated tissue occurs.
